SEQUENCE LISTING (2 sequences)

SEQ ID NO: 1 - protein, 354 aa, Artificial Sequence
  Chimeric Receptor
MGCTLSAEERAALERSKAIEKNLKEDGISAAKDVKLLLLGAGESGKSTIVKQMKIIHEDGFSGEDVKQYKPVVYSNTIQSLAAIVRAMDTLGIEYGDKERKADAKMVCDVVSRMEDTEPFSAELLSAMMRLWGDSGIQECFNRSREYQLNDSAKYYLDSLDRIGAADYQPTEQDILRTRVKTTGIVETHFTFKNLHFRLFDVGGQRSERKKWIHCFEDVTAIIFCVALSGYDQVLHEDETTNRMHESLKLFDSICNNKWFTDTSIILFLNKKDIFEEKIKKSPLTICFPEYTGPSAFTEAVAYIQAQYESKNKSAHKEIYTHVTCATDTNNIRFVFAAVKDTILQLNLKEYNLV

SEQ ID NO: 2 - DNA, 1065 bp, Artificial Sequence
  cDNA of SEQ ID NO 1
atgggatgtactctgagcgcagaggagagagccgccctcgagcggagcaaggcgattgagaaaaacctcaaagaggatggcatcagcgccgccaaagacgtgaaattactcctgctcggggctggagaatcaggaaaaagcaccattgtgaagcagatgaagatcatccatgaagatggcttctccggagaagacgtgaaacagtacaagcctgttgtctacagcaacactatccagtccctggcagccatcgtccgggccatggacactttgggcatcgaatatggtgataaggagagaaaggctgacgccaagatggtgtgtgatgtggtgagtcggatggaagacaccgagcccttctctgcagagctgctttctgccatgatgcggctctggggcgactcaggaatccaagagtgcttcaaccggtcccgggagtatcagctcaacgactctgccaaatactacctggacagcctggatcggattggggccgccgactaccagcccaccgagcaggacatcctccgaaccagggtcaaaaccactggcatcgtagaaacccacttcacattcaagaacctccacttcaggctgtttgacgtcggaggccagcgatctgaacgcaagaagtggatccattgcttcgaggacgtcacggccatcattttctgtgtcgcgctcagcggctatgaccaggtgctccacgaagacgaaaccacgaaccgcatgcacgaatccctgaagctttttgacagcatctgcaacaacaaatggttcacagacacgtccatcatcctgtttcttaacaagaaggacatatttgaagagaagatcaagaagtccccgctcaccatctgctttcctgaatatacaggccccagcgccttcacagaagccgtggcttacatccaggcccagtacgagagcaagaacaagtcagcccacaaggagatctacacccacgtcacctgcgccacggacaccaacaacatccgctttgtctttgctgccgtcaaggacaccatcctccagttgaacctgaaggagtacaatctggtctaa

(19) United States
(12) Patent Application Publication
(10) Pub. No.: US 2016/0022636 A1
(43) Pub. Date: Jan. 28, 2016

(54) PHYSIOLOGICAL LIGANDS FOR GPR139

(71) Applicant: JANSSEN PHARMACEUTICA NV, Beerse (BE)

(72) Inventors: Curt A. DVORAK, Poway, CA (US); Changlu LIU, San Diego, CA (US); Chester KUEI, San Diego, CA (US)

(21) Appl. No.: 14/775,440

(22) PCT Filed: Mar. 14, 2014
(86) PCT No.: PCT/US2014/028222

§ 371 Date: Sep. 11, 2015

Related U.S. Application Data
(60) Provisional application No. 61/782,158, filed on Mar. 14, 2013.

(51) Int. Cl. A61K 31/405; A61K 31/198; C12Q 1/68

(52) U.S. Cl. CPC A61K 31/405; C12Q 1/6883; A61K 31/198; C12Q 2600/158

(57) ABSTRACT

Provided herein are compounds capable of activating GPR139. Also provided are methods of increasing and decreasing the activity of GPR139. Methods of using the identified compounds to modulate GPR139 activity or conditions that may be affected by GPR139 activity are also disclosed.

### CROSS REFERENCE TO RELATED APPLICATIONS

This application claims priority to U.S. Application No. 61/782,158 filed on Mar. 14, 2013.

### TECHNICAL FIELD

This application relates to physiological ligands for the orphan receptor GPR139, methods of using those ligands to activate the receptor in a physiological environment, and methods of modulating neurological functions or conditions by changing the activation state of GPR139.

### BACKGROUND

G-protein coupled receptors (GPCRs) are compelling targets for drug discovery. Currently, about 30% of drugs on the market target GPCRs. Sequencing of the human genome revealed thousands of new genes, including hundreds of new G-protein coupled receptors, offering many new opportunities for drug discovery. However, these new GPCRs were identified based on their sequence and structural similarities to known GPCRs and their ligands, and thus their biological significances remain unknown. Without the ligands for these receptors, it is difficult to understand their physiological function. Furthermore, finding the ligands for the receptors will certainly help to establish assays to screen for agonists, antagonists, and modulators of the receptors.

GPR139 is an orphan G-protein coupled receptor that is predominantly expressed in the brain (Vanti et al., Biochem. Biophys. Res. Commun. 305(1):67-71 (2003); Gloriam et al., Biochim Biophys Acta. 1722(3):235-46 (2005); Matsuo et al., Biochem. Biophys. Res. Commun. 331(1):363-9 (2005)). It is coupled with Gq signaling and appears to be constitutively active when recombinantly expressed in mammalian cells (Matsuo et al., 2005). GPR139 is highly conserved among different species. For example, human, mouse and rat GPR139 protein sequences share greater than 94% identity at the amino acid level (FIG. 1). In addition, human GPR139 also shares 94% and 72% identity to a putative protein encoded by chicken and zebrafish genomes, respectively. GPR139's predominant expression in the brain and high degree of sequence homology across different species, suggest it has an important role in vertebrate physiology; however, no physiological ligand for GPR139 has been identified to date.

### SUMMARY

Provided herein are methods of activating GPR139 by contacting the receptor with L-tryptophan (L-Trp), L-phenylalanine (L-Phe), or derivatives thereof.

Also described are methods for reducing the activity of GPR139 in a subject by identifying a subject in need of reduced GPR139 activation, and administering an amount of one or more compounds sufficient to reduce the activity of GPR139 to the subject, which in turn will decrease the level of GPR139 activation relative to the native activation state.

Methods for modulating a neurological function or condition in a subject by administering an amount of one or more compounds sufficient to modulate the activity GPR139 to the subject are also described herein.

Also provided are methods for modulating a disease condition of the pancreas in a subject by administering an amount of one or more compounds sufficient to modulate the activity GPR139 to the subject.

Also provided are methods for modulating Phenylketonuria (PKU) and pituitary dysfunctions such as hyperpituitarism and hypopituitarism in a subject by administering an amount of one or more compounds sufficient to modulate the activity GPR139 to the subject.

In addition, provided herein are methods of administering any one of L-tryptophan, L-phenylalanine, tryptamine, amphetamine, D-tryptophan, D-phenylalanine, β-phenylethylamine, a compound listed in Table 4 or a derivative of any one of these compounds to a subject.

In addition the present invention provides an assay for detecting GPR139 modulation in a cell. The assay method includes detecting GPR139 activation in a cell by exposing the cell or a membrane obtained from the cell to a compound, and determining whether or not GPR139 is activated by the compound.

Described herein is chimeric G protein, designated GO2Q, and methods for using it to detect activation of GPCRs. Also described are methods for detecting GPR139 activation by co-expressing GO2Q chimeric G-protein and GPR139 in a cell by exposing the cell or a membrane obtained from the cell to a compound, and determining whether or not GPR139 is activated by the compound.

In addition, the present invention provides for an assay for screening cells recombinantly expressing GPR139. The assay method includes screening cells recombinantly expressing GPR139 with compound modulators, (such as agonists, antagonists, and allosteric modulators), either using the whole cells or cell membranes where the cell membranes harbor the recombinant GPR139 expression. The assays and methods include, but are not limited to, Ca²⁺ mobilization assays, GTPγS binding assays, radioligand binding assays, ErK phosphorylation assays, and SRE-reporter assays, using Trp, Phe as examples as stimulators. In one embodiment of the invention, since it is has been discovered that Trp and Phe serve as activators of GPR139, in one of the above mentioned assays, a compound could be added to the cell containing Trp, Phe or another activator of GPR139 and then based on the cellular response, one could determine if said added compound was an antagonist. In another aspect, the Trp and Phe ligands can be used in the above mentioned assays and methods to discover compounds which function as allosteric modulators, such allosteric modulators being compounds that bind to GPR139 to enhance or reduce the potency or efficacy of Trp, or Phe.

In addition, provided herein are assays and methods wherein GPR139 is transcribed into RNA in vitro, followed by in vitro translation into protein. The resultant protein may be used for protein/compound interactions to screen for GPR139 modulators.

### BRIEF DESCRIPTION OF THE DRAWINGS

FIG. 1: Provides an alignment of the human, mouse, and rat GPR139 amino acid sequences. The sequences shown are identical to the sequences reported in Genbank for accession numbers: AK291384 (human) (SEQ ID NO: 3), NM_{—}001024138 (mouse) (SEQ ID NO: 4), and NM_{—}001024241 (rat) (SEQ ID NO: 5). A consensus sequence (SEQ ID NO: 6) is also provided.

FIG. 2: Tryptophan, phenylalanine, and their derivatives activate GPR139. Human (A and B) or mouse (C and D) GPR139 were co-transfected with chimeric G-protein GO2Q into COS7 cells. Cell membranes from transfected cells were used in GTPγS binding studies using various ligands at different concentrations to stimulate GPR139. Membranes from COS7 cells transfected by GO2Q were used as the controls (E and F).

FIG. 3: Provides the results of experiments conducted to determine whether Trp, Phe, and their derivatives stimulate Ca²⁺ mobilization in HEK293 cells expressing GPR139 (separate graphs are shown for certain compounds tested).

FIG. 4: Shows microscopic images of HEK-293 cells transiently transfected with GPR139 in the absence (A) or presence (B) of L-Phe or L-Trp.

FIG. 5: Shows the presence of GPR142 mRNA (lanes 1 and 2) and GPR139 mRNA (lanes 4 and 5) in murine brain cells and murine pancreatic cells (Min6), respectively.

FIGS. 6 (A) and (B): Shows the detection of GPR139 in rat brain sections by ribo-probe hybridization using an 35S-labeled antisense sequence.

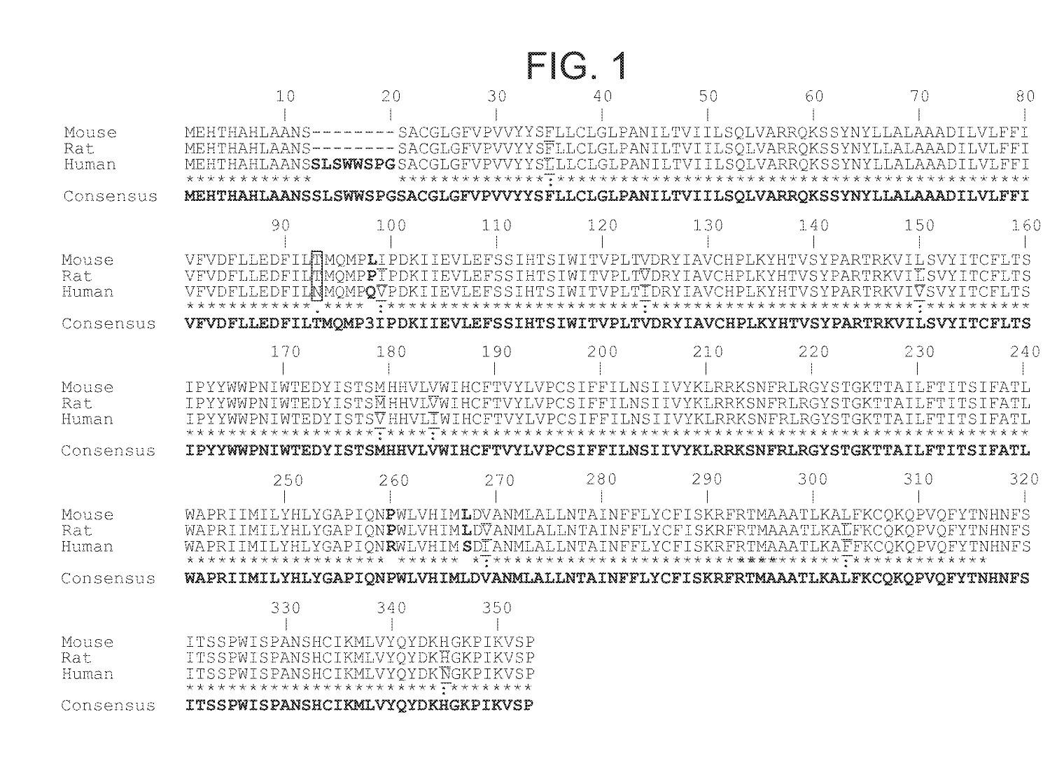

### DETAILED DESCRIPTION OF ILLUSTRATIVE EMBODIMENTS

Various terms relating to aspects of the description are used throughout the specification and claims. Such terms are to be given their ordinary meaning in the art unless otherwise indicated. Other specifically defined terms are to be construed in a manner consistent with the definitions provided herein.

The terms “Trp” and “Phe” as used herein refer to L-tryptophan (L-Trp) or D-tryptophan and (D-Trp) and L-phenylalanine (L-Phe) or D-phenylalanine (D-Phe).

“Effective amount” and amount “sufficient” are used interchangeably herein, and mean an amount or dose sufficient to generally bring about the desired therapeutic or prophylactic benefit in patients in need of such treatment for the designated disease, disorder, or condition.

Effective amounts or doses of the compounds of the present invention may be ascertained by routine methods such as modeling, dose escalation studies or clinical trials, and by taking into consideration routine factors, e.g., the mode or route of administration or drug delivery, the pharmacokinetics of the compound, the severity and course of the disease, disorder, or condition, the subject's previous or ongoing therapy, the subject's health status and response to drugs, and the judgment of the treating physician. An example of a dose is in the range of from about 0.001 to about 200 mg of compound per kg of subject's body weight per day, preferably about 0.05 to 100 mg/kg/day, or about 1 to 35 mg/kg/day, in single or divided dosage units (e.g., BID, TID, QID). For a 70-kg human, an illustrative range for a suitable dosage amount is from about 0.05 to about 7 g/day

The term “a neurological function or condition” as used herein may refer to any one of a sleep disorder (Voderholzer, U.; Guilleminault, C. (2012). “Sleep disorders”. Neurobiology of Psychiatric Disorders. Handbook of Clinical Neurology 106. pp. 527-40), depressive disorders such as major depressive disorder (Kessler R C, Nelson C, McGonagle K A, et al. (1996). Br J Psychiatry. 168(suppl 30):17-30.), treatment-resistant depression (Souery D I, Papakostas G I, Trivedi M H. J (2006) Clin Psychiatry, 67 Suppl 6:16-22.), bipolar disorder (Zimmerman M I, Martinez J H, Morgan T A, Young D, Chelminski I, Dalrymple K. (2013). J Clin Psychiatry. 74(9):880-6), schizophrenia (Buckley P F, Miller B J, Lehrer D S, Castle D J (March 2009). Schizophr Bull 35 (2): 383-402, Parkinson's Disease (Jankovic J (April 2008). J. Neurol. Neurosurg. Psychiatr. 79 (4): 368-76.), cognitive impairment, Alzheimer's Disease (Waldemar G. (2007) Eur J Neurol. 14(1):e1-26), attention deficit disorders (Lange, K W.; Reichl, S; Lange, K M.; Tucha, L; Tucha, O (2010). 2 (4): 241-255.), neurotransmitter release or absorption, short-term memory (Davelaar, E. J.; Goshen-Gottstein, Y.; Haarmann, H. J.; Usher, M.; Usher, M (2005), Psychological Review 112 (1): 3-42, long term memory (Atkinson, R. C.; Shiffrin, R. M. (1968). 2: 89-195.), or post-traumatic stress disorder (Fullerton, C. S.; Ursano, W. (2004). Am J Psychiatry 161 (8): 1370-1376.), and similar such functions and disorders.

“Pharmaceutically acceptable” means approved or approvable by a regulatory agency of the Federal or a state government or the corresponding agency in countries other than the United States, or that is listed in the U.S. Pharmacopoeia or other generally recognized pharmacopoeia for use in animals, and more particularly, in humans.

“Pharmaceutically acceptable carrier” refers to a medium that does not interfere with the effectiveness of the biological activity of the active ingredient(s), for example, compounds disclosed herein, and is not toxic to the host to which it is administered.

“Specific binding” refers to the ability of an antibody, or antigen-binding fragment, to bind to a particular biomolecule or compound with an affinity that is greater than that with which it may bind other biomolecules or compounds.

The term “subject” as used herein may refer to an animal, and preferably is a mammal such as a mouse, rat, hamster, guinea pig, rabbit, cat, dog, monkey, donkey, cow, horse, pig, and the like. Most preferably, the mammal is a human.

Provided herein are methods for activating GPR139 in a subject by identifying a subject in need of GPR139 activation, and administering an amount of one or more compounds sufficient to activate GPR139 to the subject, which in turn will increase the level of GPR139 activation relative native activation state. In some embodiments the compounds that can be administered for this purpose are provided in Table 4, such that any one of the compounds listed in Table 4 could be administered to a subject to activate GPR139. In some embodiments the compounds that can be administered for this purpose are any one of L-tryptophan, L-phenylalanine, tryptamine, amphetamine, D-tryptophan, D-phenylalanine, β-phenylethylamine or a derivative thereof. In some embodiments the compounds that can be administered for this purpose are any one of L-tryptophan, L-phenylalanine, tryptamine, amphetamine, D-tryptophan, D-phenylalanine or β-phenylethylamine. In an alternative embodiment a combination of any of the compounds listed in Table 4 could be administered to a subject to activate GPR139. More specifically, any one of L-tryptophan, L-phenylalanine, tryptamine, amphetamine, D-tryptophan, D-phenylalanine, β-phenylethylamine or a derivative thereof could be administered to a subject to activate GPR139.

Provided herein are methods for reducing the activity of GPR139 in a subject by identifying a subject in need of reduced GPR139 activation, and administering an amount of one or more compounds sufficient to reduce the activity of GPR139 to the subject, which in turn will decrease the level of GPR139 activation relative native activation state. In some embodiments the compounds that can be administered for this purpose are compounds capable of interfering with the interaction of GPR139 and any one of L-tryptophan, L-phenylalanine, tryptamine, amphetamine, D-tryptophan, D-phenylalanine or β-phenylethylamine, a compound listed in Table 4 or a derivative of any one of these compounds. In some embodiments the interfering compound may be a protein, protein fragment, or a small molecule capable of interacting with any one of L-tryptophan, L-phenylalanine, tryptamine, amphetamine, D-tryptophan, D-phenylalanine or β-phenylethylamine, a compound listed in Table 4 or a derivative of any one of these compounds, or GPR139. In some embodiments the interfering compound may be an antibody, or an antibody fragment, that specifically binds to GPR139 and inhibits or prevents its interaction with an activating compound. In some embodiments the interfering compound may be an antibody, or an antibody fragment, that specifically binds to L-tryptophan, L-phenylalanine, tryptamine, amphetamine, D-tryptophan, D-phenylalanine or β-phenylethylamine, a compound listed in Table 4 or a derivative of any one of these compounds and inhibits or prevents its interaction with GPR139. In some embodiments the interfering compound may be an antibody, or an antibody fragment, that specifically binds to L-tryptophan, L-phenylalanine, tryptamine, amphetamine, D-tryptophan, D-phenylalanine or β-phenylethylamine, and inhibits or prevents its interaction with GPR139.

Also described herein are methods for modulating a neurological function or condition in a subject by administering an amount of one or more compounds sufficient to modulate the activity GPR139 to the subject. Also described herein are methods for modulating a neurological function or condition by contacting a cell, such as a brain cell, in a subject by administering an amount of one or more compounds sufficient to modulate the activity GPR139 to the subject. Examples of such brain cells include but are not limited to neuronal cells, microglial cells, astrocytes and oligodendrial cells. In some embodiments the neurological function or condition may be modulated by increasing the activity of GPR139. In some embodiments a neurological function or condition may be modulated by increasing the activity of GPR139 by administering to a subject any one of the compounds listed in Table 4 in order to activate GPR139. In some embodiments a neurological function or condition may be modulated by increasing the activity of GPR139 by administering to a subject any one of the compounds listed in Table 4 in order to increase the activation level of GPR139. In some embodiments a neurological function or condition may be modulated by increasing the activity of GPR139 by administering to a subject any one of L-tryptophan, L-phenylalanine, tryptamine, amphetamine, D-tryptophan, D-phenylalanine, β-phenylethylamine or a derivative thereof in order to activate GPR139. In some embodiments a neurological function or condition may be modulated by increasing the activity of GPR139 by administering to a subject any one of L-tryptophan, L-phenylalanine, tryptamine, amphetamine, D-tryptophan, D-phenylalanine, β-phenylethylamine or a derivative thereof in order to increase the activation level of GPR139. In some embodiments the neurological function or condition may be modulated by decreasing the activity of GPR139. In some embodiments a neurological function or condition may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine, a compound listed in Table 4 or a derivative of any one of these compounds. In some embodiments a neurological function or condition may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine, or a compound listed in Table 4. In some embodiments a neurological function or condition may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine. In some embodiments the interfering compound may be a protein, protein fragment, or a small molecule capable of interacting with any one of L-tryptophan, L-phenylalanine, tryptamine, amphetamine, D-tryptophan, D-phenylalanine or β-phenylethylamine, a compound listed in Table 4 or a derivative of any one of these compounds, or GPR139. In some embodiments the interfering compound may be an antibody, or an antibody fragment, that specifically binds to GPR139 and inhibits or prevents its interaction with an activating compound. In some embodiments the interfering compound may be an antibody, or an antibody fragment, that specifically binds to L-tryptophan, L-phenylalanine, tryptamine, amphetamine, D-tryptophan, D-phenylalanine or β-phenylethylamine, a compound listed in Table 4 or a derivative of any one of these compounds and inhibits or prevents its interaction with GPR139. In some embodiments the interfering compound may be an antibody, or an antibody fragment, that specifically binds to L-tryptophan, L-phenylalanine, tryptamine, amphetamine, D-tryptophan, D-phenylalanine or β-phenylethylamine, and inhibits or prevents its interaction with GPR139.

Described herein are methods for modulating a disease condition of the pancreas in a subject by administering an amount of one or more compounds sufficient to modulate the activity of GPR139 to the subject. In some embodiments the disease condition of the pancreas may be modulated by increasing the activity of GPR139. In some embodiments a disease condition of the pancreas may be modulated by increasing the activity of GPR139 by administering to a subject any one of the compounds listed in Table 4 in order to activate GPR139. In some embodiments a disease condition of the pancreas may be modulated by increasing the activity of GPR139 by administering to a subject any one of the compounds listed in Table 4 in order to increase the activation level of GPR139. In some embodiments a disease condition of the pancreas may be modulated by increasing the activity of GPR139 by administering to a subject any one of L-tryptophan, L-phenylalanine, tryptamine, amphetamine, D-tryptophan, D-phenylalanine, β-phenylethylamine or a derivative thereof in order to activate GPR139. In some embodiments a disease condition of the pancreas may be modulated by increasing the activity of GPR139 by administering to a subject any one of L-tryptophan, L-phenylalanine, tryptamine, amphetamine, D-tryptophan, D-phenylalanine, β-phenylethylamine or a derivative thereof in order to increase the activation level of GPR139. In some embodiments the disease condition of the pancreas may be modulated by decreasing the activity of GPR139. In some embodiments a disease condition of the pancreas may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine, a compound listed in Table 4 or a derivative of any one of these compounds. In some embodiments a disease condition of the pancreas may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine, or a compound listed in Table 4. In some embodiments a disease condition of the pancreas may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine. In some embodiments the interfering compound may be a protein, protein fragment, or a small molecule capable of interacting with any one of L-tryptophan, L-phenylalanine, tryptamine, amphetamine, D-tryptophan, D-phenylalanine or β-phenylethylamine, a compound listed in Table 4 or a derivative of any one of these compounds, or GPR139. In some embodiments the interfering compound may be an antibody, or an antibody fragment, that specifically binds to GPR139 and inhibits or prevents its interaction with an activating compound. In some embodiments the interfering compound may be an antibody, or an antibody fragment, that specifically binds to L-tryptophan, L-phenylalanine, tryptamine, amphetamine, D-tryptophan, D-phenylalanine or β-phenylethylamine, a compound listed in Table 4 or a derivative of any one of these compounds and inhibits or prevents its interaction with GPR139. In some embodiments the interfering compound may be an antibody, or an antibody fragment, that specifically binds to L-tryptophan, L-phenylalanine, tryptamine, amphetamine, D-tryptophan, D-phenylalanine or β-phenylethylamine, and inhibits or prevents its interaction with GPR139.

Described herein are methods for modulating Phenylketonuria (PKU) and pituitary dysfunctions such as hyperpituitarism and hypopituitarism in a subject by administering an amount of one or more compounds sufficient to modulate the activity GPR139 to the subject. Phenylketonuria (PKU) is an autosomal recessive genetic disorder characterized by a mutation in the gene for the hepatic enzyme phenylalanine hydroxylase (PAH), rendering it nonfunctional. This enzyme is necessary to metabolize the amino acid phenylalanine (Phe). Patients with PKU have abnormally high concentration of Phe in the plasma and tissues and untreated patients will have growth retardation and brain related disorders, such as intellectual disability, seizures, and other serious medical problems (Centerwall, S. A. & Centerwall, W. R. (2000). Pediatrics 105 (1 Pt 1): 89-103.; Blau N, van Spronsen, Levy H L: Lancet 2010, 376:1417-1427.). Pituitary is a small but very important organ in the body. It produces or controls many hormone secretions. The abnormally high Phe in the body could cause over stimulation of its receptor, GPR139, which is highly expressed in the brain and pituitary. Therefore GPR139 may play roles in PKU and pituitary disorders that cause abnormal hormone secretions, such as Hyperpituitarism (Colao, A, Loche, S, Cappabianca, P. The Endocrinologist. 2000; 10:314-27; Colao, A, Lombardi, G. Lancet. Oct. 31, 1998; 352(9138):1455-61) or Hypopituitarism (Schneider H J, Aimaretti G, Kreitschmann-Andermahr I, Stalla G K, Ghigo E (April 2007). Lancet 369 (9571): 1461-70.).

In some embodiments, Phenylketonuria (PKU) and pituitary dysfunctions such as hyperpituitarism and hypopituitarism may be modulated by increasing the activity of GPR139. An additional embodiment of the invention is a method of modulating Phenylketonuria (PKU), and pituitary dysfunctions such as hyperpituitarism and hypopituitarism, comprising contacting a cell of a subject, (such as brain cell, pancreatic cell, pituitary cell or other type of cell) with one or more compounds sufficient to modulate the activity of GPR139 to the subject. In some embodiments, Phenylketonuria (PKU) and pituitary dysfunctions such as hyperpituitarism and hypopituitarism may be modulated by increasing the activity of GPR139 by administering to a subject any one of the compounds listed in Table 4 in order to activate GPR139. In some embodiments, Phenylketonuria (PKU) and pituitary dysfunctions such as hyperpituitarism and hypopituitarism may be modulated by increasing the activity of GPR139 by administering to a subject any one of the compounds listed in Table 4 in order to increase the activation level of GPR139. In some embodiments, Phenylketonuria (PKU) and pituitary dysfunctions such as hyperpituitarism and hypopituitarism may be modulated by increasing the activity of GPR139 by administering to a subject any one of L-tryptophan, L-phenylalanine, tryptamine, amphetamine, D-tryptophan, D-phenylalanine, (β-phenylethylamine or a derivative thereof in order to activate GPR139. In some embodiments, Phenylketonuria (PKU) and pituitary dysfunctions such as hyperpituitarism and hypopituitarism may be modulated by increasing the activity of GPR139 by administering to a subject any one of L-tryptophan, L-phenylalanine, tryptamine, amphetamine, D-tryptophan, D-phenylalanine, β-phenylethylamine or a derivative thereof in order to increase the activation level of GPR139. In some embodiments, Phenylketonuria (PKU) and pituitary dysfunctions such as hyperpituitarism and hypopituitarism may be modulated by decreasing the activity of GPR139. In some embodiments, Phenylketonuria (PKU) and pituitary dysfunctions such as hyperpituitarism and hypopituitarism may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine, a compound listed in Table 4 or a derivative of any one of these compounds. In some embodiments, Phenylketonuria (PKU) and pituitary dysfunctions such as hyperpituitarism and hypopituitarism may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine, or a compound listed in Table 4. In some embodiments, Phenylketonuria (PKU), and pituitary dysfunctions such as hyperpituitarism and hypopituitarism may be modulated by decreasing the activity of GPR139 by administering to a subject a compound capable of interfering with the interaction of GPR139 and any one of L-tryptophan, L-phenylalanine, tryptamine, amphetamine, D-tryptophan, D-phenylalanine or β-phenylethylamine. In some embodiments, the interfering compound may be a protein, protein fragment, or a small molecule capable of interacting with any one of L-tryptophan, L-phenylalanine, tryptamine, amphetamine, D-tryptophan, D-phenylalanine or β-phenylethylamine, a compound listed in Table 4 or a derivative of any one of these compounds, or GPR139. In some embodiments, the interfering compound may be an antibody, or an antibody fragment, that specifically binds to GPR139 and inhibits or prevents its interaction with an activating compound. In some embodiments, the interfering compound may be an antibody, or an antibody fragment, that specifically binds to L-tryptophan, L-phenylalanine, tryptamine, amphetamine, D-tryptophan, D-phenylalanine or β-phenylethylamine, a compound listed in Table 4 or a derivative of any one of these compounds and inhibits or prevents its interaction with GPR139. In some embodiments, the interfering compound may be an antibody, or an antibody fragment, that specifically binds to L-tryptophan, L-phenylalanine, tryptamine, amphetamine, D-tryptophan, D-phenylalanine or β-phenylethylamine, and inhibits or prevents its interaction with GPR139.

An additional embodiment of the invention is wherein the compound is administered locally to the subject.

An additional embodiment of the invention is wherein the compound is administered orally or intravenously to the subject.

An additional embodiment of the invention is wherein the compound is administered locally to a subject having a neuronal cell.

An additional embodiment of the invention is wherein the compound is administered orally or intravenously to a subject having a neuronal cell.

An additional embodiment of the invention is a method for detecting GPR139 activation comprising, co-expressing GO2Q chimeric G-protein and GPR139 in a cell, exposing the cell or a membrane obtained from the cell to a compound, and determining whether or not GPR139 is activated by the compound.

Also provided herein are methods of administering any one of L-tryptophan, L-phenylalanine, tryptamine, amphetamine, D-tryptophan, D-phenylalanine β-phenylethylamine, a compound listed in Table 4 or a derivative of any one of these compounds to a subject. Any one of the compounds described herein may be administered to a subject orally in any acceptable dosage form such as capsules, tablets, aqueous suspensions, solutions or the like. Any one of the compounds may also be administered to a subject parenterally including but not limited to: subcutaneous, intravenous, intramuscular, intra-articular, intra-synovial, intrasternal, intranasal, topically, intrathecal, intrahepatic, intralesional, and intracranial injection or infusion techniques. Alternatively, any one of the compounds can be administered to a subject intravenously or intraperitoneally, for example, by injection. Administration of the compounds described herein to a subject may be carried out using a pharmaceutically acceptable formulation that includes the any one of the compounds for activating or inhibiting the activation of GPR139 described herein. Such formulations may also include a pharmaceutically acceptable carrier, as are commonly known in the art.

### Chimeric G-Protein GO2Q

Historically, the Gi, Go coupled GPCRs offer a much greater signal-to-noise ratio in GTPγS binding studies. For Gs and Gq coupled GPCRs, although ligand-stimulated specific signals can be detected in GTPγS binding assay, the signal-to-noise ratios are often very small. Described herein is a chimeric G-protein that can be co-expressed with a GPCR in a cell to increase the signal-to-noise ratio in a GTPγS binding assay and thereby allow for detection of GPCR activity. In some embodiments the chimeric G-protein has an N-terminus from a Go2 protein and a C-terminus from a Gq protein in about any one of the ratio combinations shown in Table 1.

TABLE 1
Proportions of a Go2/Gq chimeric G-protein

| Portion of total chimeric | Portion of total chimeric |
|---|---|
| G-protein from the N- | G-protein from the C- |
| terminus from Go2 protein | terminus from Gq protein |
| 95 | 5 |
| 90 | 10 |
| 85 | 15 |
| 80 | 20 |
| 75 | 25 |
| 70 | 30 |
| 65 | 35 |
| 60 | 40 |
| 55 | 45 |
| 50 | 50 |
| 45 | 55 |
| 40 | 60 |
| 35 | 65 |
| 30 | 70 |
| 25 | 75 |
| 20 | 80 |
| 15 | 85 |
| 10 | 90 |
| 5 | 95 |

In some embodiments the chimeric G-protein has the amino acid sequence: MGCTLSAEERAALERSKAIEKNLKEDGISAAKDVKLLLLGAGESGKSTIVKQMKIIHED GFSGEDVKQYKPVVYSNTIQSLAAIVRAMDTLGIEYGDKERKADAKMVCDVVSRMED TEPFSAELLSAMMRLWGDSGIQECFNRSREYQLNDSAKYYLDSLDRIGAADYQPTEQDI LRTRVKTTGIVETHFTFKNLHFRLFDVGGQRSERKKWIHCFEDVTAIIFCVALSGYDQVL HEDETTNRMHESLKLFDSICNNKWFTDTSIILFLNKKDIFEEKIKKSPLTICFPEYTGPSAF TEAVAYIQAQYESKNKSAHKEIYTHVTCATDTNNIrfvfaavkdtilqlnlkeynlv (SEQ ID NO:1), where the capitalized residues are from the N-terminus from Go2 protein and the lower-case residues are from the C-terminus from Gq protein. The chimeric G-protein GO2Q disclosed herein consists of the sequence of SEQ ID NO:1. In some aspects the sequence of SEQ ID NO:1 is encoded by the following DNA sequence: ATGGGATGTACTCTGAGCGCAGAGGAGAGAGCCGCCCTCGAGCGGAGCAAGGCGA TTGAGAAAAACCTCAAAGAGGATGGCATCAGCGCCGCCAAAGACGTGAAATTACTC CTGCTCGGGGCTGGAGAATCAGGAAAAAGCACCATTGTGAAGCAGATGAAGATCAT CCATGAAGATGGCTTCTCCGGAGAAGACGTGAAACAGTACAAGCCTGTTGTCTACA GCAACACTATCCAGTCCCTGGCAGCCATCGTCCGGGCCATGGACACTTTGGGCATC GAATATGGTGATAAGGAGAGAAAGGCTGACGCCAAGATGGTGTGTGATGTGGTGA GTCGGATGGAAGACACCGAGCCCTTCTCTGCAGAGCTGCTTTCTGCCATGATGCGGC TCTGGGGCGACTCAGGAATCCAAGAGTGCTTCAACCGGTCCCGGGAGTATCAGCTC AACGACTCTGCCAAATACTACCTGGACAGCCTGGATCGGATTGGGGCCGCCGACTA CCAGCCCACCGAGCAGGACATCCTCCGAACCAGGGTCAAAACCACTGGCATCGTAG AAACCCACTTCACATTCAAGAACCTCCACTTCAGGCTGTTTGACGTCGGAGGCCAGC GATCTGAACGCAAGAAGTGGATCCATTGCTTCGAGGACGTCACGGCCATCATTTTCT GTGTCGCGCTCAGCGGCTATGACCAGGTGCTCCACGAAGACGAAACCACGAACCGC ATGCACGAATCCCTGAAGCTTTTTGACAGCATCTGCAACAACAAATGGTTCACAGA CACGTCCATCATCCTGTTTCTTAACAAGAAGGACATATTTGAAGAGAAGATCAAGA AGTCCCCGCTCACCATCTGCTTTCCTGAATATACAGGCCCCAGCGCCTTCACAGAAG CCGTGGCTTACATCCAGGCCCAGTACGAGAGCAAGAACAAGTCAGCCCACAAGGA GATCTACACCCACGTCACCTGCGCCACGGACACCAACAACATCcgctttgtattgctgccgtcaa ggacaccatcctccagttgaacctgaaggagtacaatctggtctaa (SEQ ID NO: 2), where the capitalized residues represent the sequence that encodes the N-terminus from Go2 protein and the lower-case residues represent the sequence that encodes the C-terminus from Gq protein.

Also described herein is a method for detecting activation of a GPCR using a chimeric Go2/Gq G-protein. The described method capitalizes on the preferred signal-to-noise ratio provided by chimeric G-proteins of this sort. In some embodiments the described method is conducted by co-expressing a chimeric Go2/Gq G-protein with a GPCR in a cell, contacting the cell with a compound, and determining whether the compound activates the GPCR. In some embodiments the method involves co-expressing a chimeric Go2/Gq G-protein with a GPCR in a cell, contacting the cell with a compound, and determining whether the compound activates the GPCR, where the chimeric Go2/Gq G-protein has an N-terminus from a Go2 protein and a C-terminus from a Gq protein in about any one of the ratio combinations shown in Table 1. In some embodiments the method involves co-expressing a chimeric Go2/Gq G-protein with a GPCR in a cell, contacting the cell with a compound, and determining whether the compound activates the GPCR, where the chimeric Go2/Gq G-protein has the amino acid sequence of SEQ ID NO: 1. In some embodiments the method involves co-expressing a chimeric Go2/Gq G-protein with a GPCR in a cell, contacting the cell with a compound, and determining whether the compound activates the GPCR, where the chimeric Go2/Gq G-protein is GO2Q. In some embodiments the described method is conducted by co-expressing a chimeric Go2/Gq G-protein with a GPCR in a cell, contacting the cell with a compound, and determining whether the compound activates the GPCR, where the GPCR is GPR139. In some embodiments the method involves co-expressing a chimeric Go2/Gq G-protein with a GPCR in a cell, contacting the cell with a compound, and determining whether the compound activates the GPCR, where the chimeric Go2/Gq G-protein has an N-terminus from a Go2 protein and a C-terminus from a Gq protein in about any one of the ratio combinations shown in Table 1 and the GPCR is GPR139. In some embodiments the method involves co-expressing a chimeric Go2/Gq G-protein with a GPCR in a cell, contacting the cell with a compound, and determining whether the compound activates the GPCR, where the chimeric Go2/Gq G-protein has the amino acid sequence of SEQ ID NO: 1 and the GPCR is GPR139. In some embodiments the method involves co-expressing a chimeric Go2/Gq G-protein with a GPCR in a cell, contacting the cell with a compound, and determining whether the compound activates the GPCR, where the chimeric Go2/Gq G-protein is GO2Q and the GPCR is GPR139.

In some embodiments the method can be carried out with a cell membrane obtained from a cell co-expressing a chimeric Go2/Gq G-protein and a GPCR in a cell. In some embodiments the described method is conducted by co-expressing a chimeric Go2/Gq G-protein with a GPCR in a cell, isolating a segment of the cell membrane of the cell, contacting the cell membrane with a compound, and determining whether the compound activates the GPCR. In some embodiments the method involves co-expressing a chimeric Go2/Gq G-protein with a GPCR in a cell, isolating a segment of the cell membrane of the cell, contacting the cell membrane with a compound, and determining whether the compound activates the GPCR, where the chimeric Go2/Gq G-protein has an N-terminus from a Go2 protein and a C-terminus from a Gq protein in about any one of the ratio combinations shown in Table 1. In some embodiments the method involves co-expressing a chimeric Go2/Gq G-protein with a GPCR in a cell, isolating a segment of the cell membrane of the cell, contacting the cell membrane with a compound, and determining whether the compound activates the GPCR, where the chimeric Go2/Gq G-protein has the amino acid sequence of SEQ ID NO: 1. In some embodiments the method involves co-expressing a chimeric Go2/Gq G-protein with a GPCR in a cell, isolating a segment of the cell membrane of the cell, contacting the cell membrane with a compound, and determining whether the compound activates the GPCR, where the chimeric Go2/Gq G-protein is GO2Q. In some embodiments the described method is conducted by co-expressing a chimeric Go2/Gq G-protein with a GPCR in a cell, isolating a segment of the cell membrane of the cell, contacting the cell membrane with a compound, and determining whether the compound activates the GPCR, where the GPCR is GPR139. In some embodiments the method involves co-expressing a chimeric Go2/Gq G-protein with a GPCR in a cell, isolating a segment of the cell membrane of the cell, contacting the cell membrane with a compound, and determining whether the compound activates the GPCR, where the chimeric Go2/Gq G-protein has an N-terminus from a Go2 protein and a C-terminus from a Gq protein in about any one of the ratio combinations shown in Table 1 and the GPCR is GPR139. In some embodiments the method involves co-expressing a chimeric Go2/Gq G-protein with a GPCR in a cell, isolating a segment of the cell membrane of the cell, contacting the cell membrane with a compound, and determining whether the compound activates the GPCR, where the chimeric Go2/Gq G-protein has the amino acid sequence of SEQ ID NO: 1 and the GPCR is GPR139. In some embodiments the method involves co-expressing a chimeric Go2/Gq G-protein with a GPCR in a cell, isolating a segment of the cell membrane of the cell, contacting the cell membrane with a compound, and determining whether the compound activates the GPCR, where the chimeric Go2/Gq G-protein is GO2Q and the GPCR is GPR139.

The described methods for assessing activation of GPCR using a chimeric Go2/Gq G-protein expressed in the same cell can make use of a variety of cell types. In some embodiments, the described method can be carried out using a mammalian cell line modified to co-express a chimeric Go2/Gq G-protein with a GPCR of interest. In some embodiments the cell line used for the described method may be a fibroblast cell, a kidney cell, a monkey kidney cell (such as a COS cell) or other similar type of cell commonly used to assess GPCR activity. IN addition, cells that natively express a GPCR of interest could be modified to express a chimeric Go2/Gq G-protein in order to better assess GPRC activation. In some embodiments the cell modified to co-express a chimeric Go2/Gq G-protein with a GPCR of interest may be modified to transiently express either a chimeric Go2/Gq G-protein or a GPCR of interest. In some embodiments the cell modified to co-express a chimeric Go2/Gq G-protein with a GPCR of interest may be modified to transiently express both a chimeric Go2/Gq G-protein and a GPCR of interest. In some embodiments the cell modified to co-express a chimeric Go2/Gq G-protein with a GPCR of interest may be modified to stably express either a chimeric Go2/Gq G-protein or a GPCR of interest. In some embodiments the cell modified to co-express a chimeric Go2/Gq G-protein with a GPCR of interest may be modified to stably express a chimeric Go2/Gq G-protein and a GPCR of interest. In one embodiment a mammalian cell line may be modified to stably express a chimeric Go2/Gq G-protein to allow for further modification by either transient or stable expression of any GPCR of interest to produce a cell for use in the described method.

In some embodiments, the described method can be carried out using a mammalian cell line modified to co-express a chimeric Go2/Gq G-protein described in Table 1 with a GPCR of interest. In some embodiments the cell modified to co-express a chimeric Go2/Gq G-protein described in Table 1 with a GPCR of interest may be modified to transiently express either a chimeric Go2/Gq G-protein described in Table 1 or a GPCR of interest. In some embodiments the cell modified to co-express a chimeric Go2/Gq G-protein with a GPCR of interest may be modified to transiently express both a chimeric Go2/Gq G-protein described in Table 1 and a GPCR of interest. In some embodiments the cell modified to co-express a chimeric Go2/Gq G-protein with a GPCR of interest may be modified to stably express either a chimeric Go2/Gq G-protein described in Table 1 or a GPCR of interest. In some embodiments the cell modified to co-express a chimeric Go2/Gq G-protein with a GPCR of interest may be modified to stably express a chimeric Go2/Gq G-protein described in Table 1 and a GPCR of interest. In one embodiment a mammalian cell line may be modified to stably express a chimeric Go2/Gq G-protein described in Table 1 to allow for further modification by either transient or stable expression of any GPCR of interest to produce a cell for use in the described method.

In some embodiments, the described method can be carried out using a mammalian cell line modified to co-express a chimeric Go2/Gq G-protein having the amino acid sequence of SEQ ID NO: 1 and a GPCR of interest. In some embodiments the cell modified to co-express a chimeric Go2/Gq G-protein having the amino acid sequence of SEQ ID NO: 1 with a GPCR of interest may be modified to transiently express either a chimeric Go2/Gq G-protein having the amino acid sequence of SEQ ID NO: 1 or a GPCR of interest. In some embodiments the cell modified to co-express a chimeric Go2/Gq G-protein with a GPCR of interest may be modified to transiently express both a chimeric Go2/Gq G-protein having the amino acid sequence of SEQ ID NO: 1 and a GPCR of interest. In some embodiments the cell modified to co-express a chimeric Go2/Gq G-protein with a GPCR of interest may be modified to stably express either a chimeric Go2/Gq G-protein having the amino acid sequence of SEQ ID NO: 1 or a GPCR of interest. In some embodiments the cell modified to co-express a chimeric Go2/Gq G-protein with a GPCR of interest may be modified to stably express a chimeric Go2/Gq G-protein having the amino acid sequence of SEQ ID NO: 1 and a GPCR of interest. In one embodiment a mammalian cell line may be modified to stably express a chimeric Go2/Gq G-protein having the amino acid sequence of SEQ ID NO: 1 to allow for further modification by either transient or stable expression of any GPCR of interest to produce a cell for use in the described method.

The following examples are provided to describe the embodiments described herein with greater detail. They are intended to illustrate, not to limit, the embodiments.

### Example 1

### Tryptophan, Phenylalanine, and their Derivatives Activate GPR139

In order identify the ligands for GPR139 two improvements in the detection assay were made to allow for the ligands to be detected. First, we chose to use a GTPγS binding assay instead of using live cultured cells. The GTPγS binding assay is a cell free system that uses the cell membrane of the cell(s) of interest rather than the live cell(s). One advantage of this assay is that potential ligands present in the cell culture media are washed away when the cell membrane is prepared. This reduces the basal level of signal and increase the chance of detecting receptor ligands when assessing compounds that activate the receptor. Second, a GO2Q chimeric G-protein was created to be co-expressed with GPR139 in order to increase the signal-to-noise ratio in the GTPγS binding assay. Historically, the Gi, Go coupled GPCRs offer much greater signal-to-noise ratio in GTPγS binding studies. For Gs and Gq coupled GPCRs, although ligand-stimulated specific signals can be detected in a GTPγS binding assay, the signal-to-noise ratios are often very small. To address this a chimeric GO2-Gq chimeric G-protein, GO2Q, with the N-terminus from Go2 protein and the C-terminus from Gq protein. This chimeric G-protein, when co-expressed with Gq-coupled GPCRs, for instance histamine H1 receptor, was able to increase the signal-to-noise ratio to 3:1 when histamine was used as the ligand.

To test for compounds that activate GPR139, different amino acids and their derivatives (Table 2) were tested as ligands in the GTPγS binding assay using cell membranes from COS7 cells co-expressing GPR139 and GO2Q. Cell membrane from COS7 cells expressing GO2Q alone or co-expressing other GPCRs were also tested in GTPγS binding assay in parallel as controls. Each compound was tested in triplicate. The results show L-tryptophan (Trp) and L-phenylalanine (L-Phe) are able to activate GPR139 at the concentration tested (1 mM). In addition, a few tryptophan and phenylalanine derivatives, including tryptamine, β-phenylethylamine, and amphetamine also activated GPR139 at a 1 mM concentration in the GTPγS binding assay (Table 3, lower panel, positive results are bolded and correspond to the position of compounds shown in Table 2). The activation of GPR139 by L-Trp and L-Phe and their derivatives appeared to be specific since in the same assay, these compounds did not activate membranes from cells expressing GO2Q alone (Table 3, upper panel) or from cells co-expressing GO2Q and other GPCRs such as GPR21, GPR52, GPCR119, GPCR132, GPCR134, or GPR182 (data not shown).

TABLE 2
Compounds tested for activating of GPR139

| Ligand plate 1 mM for final concentration | | | | | | | | | | | |
|---|---|---|---|---|---|---|---|---|---|---|---|
| 1 | 2 | 3 | 4 | 5 | 6 | 7 | 8 | 9 | 10 | 11 | 12 |

| | | | | |
|---|---|---|---|---|
| A | Ala | Arg | Asn | Asp |
| B | Cys | Cystine | Gln | Glu |
| C | Gly | His | Ile | Met |
| D | Leu | Lys | Phe | Pro |
| E | Ser | Thr | Trp | Tyr |
| F | Val | Adrenaline | DA | HA |
| G | Octopamine | b-phenylethylamine | Seretonin | Tyramine |
| H | Tryptamine | Amphetamine | HO-Pro | Buffer |

TABLE 3
Activation levels of GPR139 by amino acids and their derivatives

| | | | | | | | | | | | |
|---|---|---|---|---|---|---|---|---|---|---|---|
| 1 | 2 | 3 | 4 | 5 | 6 | 7 | 8 | 9 | 10 | 11 | 12 |

Ct-GO2Q

| | | | | | | | | | | | | |
|---|---|---|---|---|---|---|---|---|---|---|---|---|
| A | 1903 | 2049 | 2139 | 2171 | 2131 | 2255 | 2195 | 2142 | 2133 | 2177 | 2137 | 2132 |
| B | 1928 | 2132 | 2114 | 2242 | 2149 | 2229 | 2181 | 2327 | 2336 | 2242 | 2310 | 2311 |
| C | 1936 | 2133 | 2289 | 2308 | 2259 | 2197 | 2266 | 2222 | 2253 | 2173 | 2307 | 2276 |
| D | 2194 | 2213 | 2167 | 2400 | 2138 | 2041 | 2190 | 2215 | 2345 | 2256 | 2235 | 2329 |
| E | 1975 | 2036 | 2164 | 2210 | 2213 | 2008 | 2172 | 2234 | 2272 | 2192 | 2191 | 2186 |
| F | 2072 | 2083 | 1977 | 2240 | 2117 | 2045 | 2250 | 2213 | 2319 | 2186 | 2204 | 2199 |
| G | 2087 | 2140 | 2142 | 2334 | 2065 | 2024 | 2356 | 2154 | 2332 | 2132 | 2277 | 2369 |
| H | 2025 | 2048 | 2088 | 2331 | 2132 | 2001 | 2166 | 2152 | 2189 | 2200 | 2247 | 2259 |

GPR139-GO2Q

| | | | | | | | | | | | | |
|---|---|---|---|---|---|---|---|---|---|---|---|---|
| A | 2363 | 2348 | 2439 | 2806 | 2783 | 2528 | 2806 | 2483 | 2547 | 2758 | 2922 | 2925 |
| B | 2168 | 2458 | 2336 | 2501 | 2652 | 2258 | 2583 | 2525 | 2766 | 2847 | 2837 | 2841 |
| C | 2560 | 2505 | 2414 | 2640 | 2499 | 2499 | 2760 | 2542 | 2903 | 2657 | 2829 | 2802 |
| D | 2621 | 2544 | 2437 | 2433 | 2617 | 2589 | 8697 | 6925 | 8417 | 2719 | 2657 | 2786 |
| E | 2605 | 2458 | 2545 | 2547 | 2507 | 2558 | 8141 | 9121 | 9149 | 2752 | 2715 | 2921 |
| F | 2632 | 2689 | 2533 | 2721 | 2624 | 2670 | 2688 | 2825 | 2708 | 2737 | 2863 | 2899 |
| G | 2554 | 2675 | 2831 | 6705 | 7099 | 6634 | 2839 | 2852 | 2920 | 2883 | 2932 | 2873 |
| H | 6757 | 7686 | 7742 | 7242 | 6744 | 7263 | 2825 | 2797 | 3109 | 2819 | 2769 | 2970 |

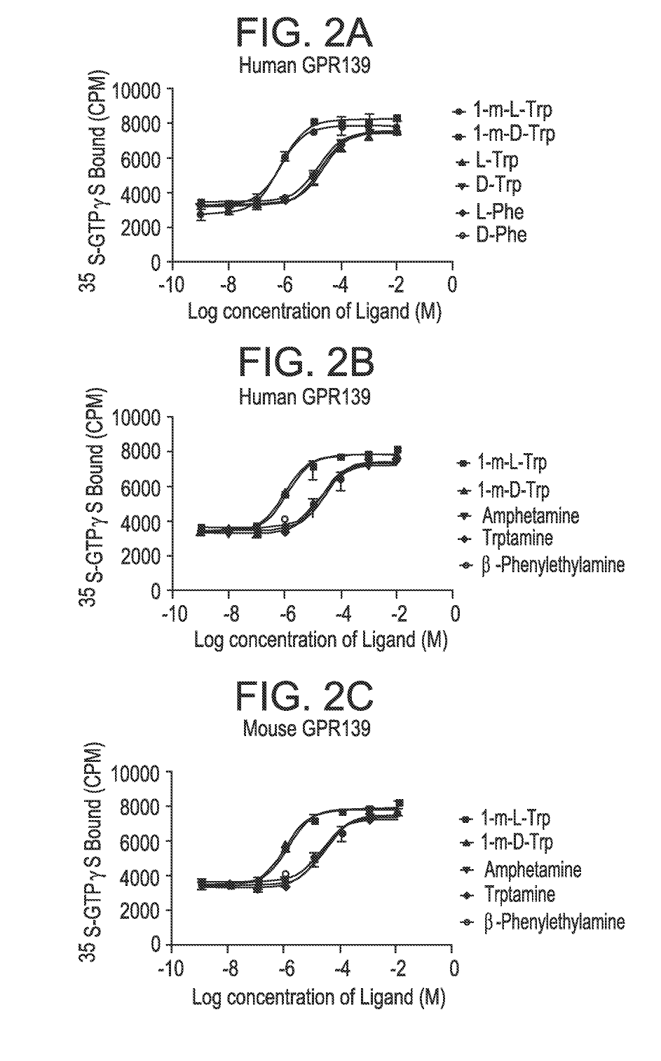

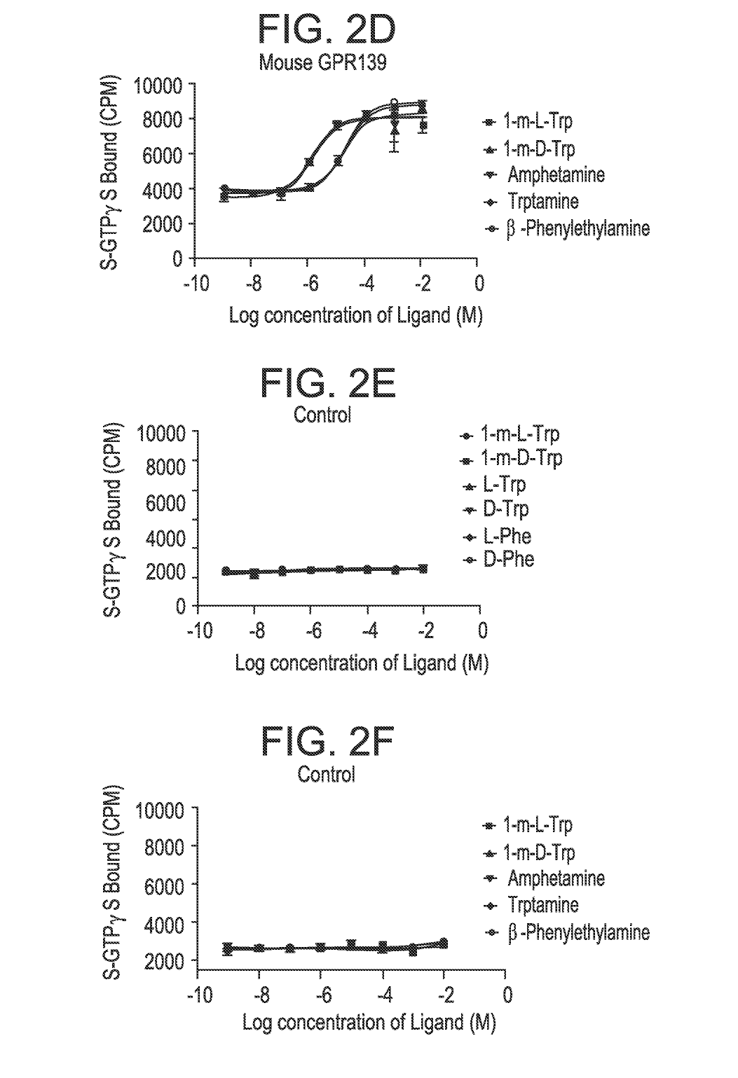

To assess the manner in which L-Trp and L-Phe and their derivatives including L-Trp, D-Trp, 1-methy-L-Trp, 1-methy-D-Try, L-Phe, D-Phe, Tryptamine, β-Phenylethylamine, and amphetamine activated GPR139, dose response studies were performed for activation of GPR139. The results show that all these compounds specifically activate GPR139 in a dose-responsive manner (FIG. 2).

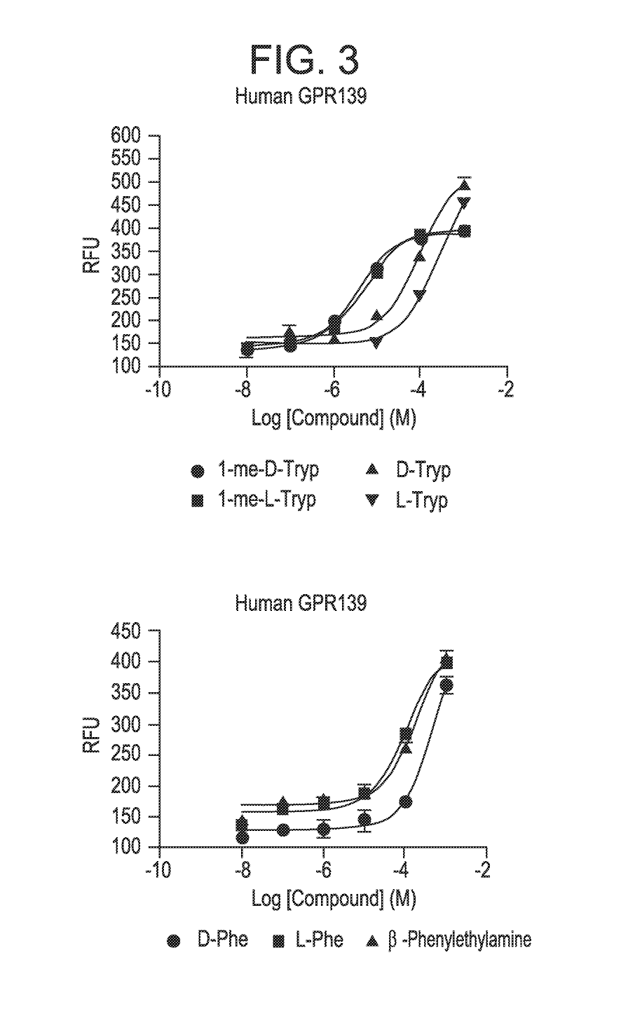

The active compounds were also tested in a calcium mobilization assay (FLIPR® assay). For these experiments human GPR139 was transiently expressed in HEK293 cells and calcium mobilization was stimulated using L-Trp, L-Phe or their derivatives. The results showed that L-Trp, L-Phe, and their derivatives also specifically stimulate calcium mobilization (FIG. 3). For GPR139 expressing cells, L-Trp and L-Phe demonstrated EC50 (half maximal effective concentration) values from 100-200 μM. Similar to GTPγS binding studies, 1-methyl-L-Trp, and 1-methyl-D-Trp demonstrated higher potencies. The concentrations of L-Trp and L-Phe in many cell culture media (for instance DMEM is about 80 μM and 750 μM respectively) are close to the EC₅₀ values of L-Trp and L-Phe for GPR139. When GPR139 is recombinantly expressed in mammalian cells and cultured in these media, the accumulated GPR139 agonist concentrations in the media would be significantly above the EC₅₀ values of L-Trp and L-Phe, therefore the receptor, GPR139, would be constantly activated in the tissue culture conditions. This may explain why GPR139 appeared constitutively active when recombinantly expressed in mammalian cells.

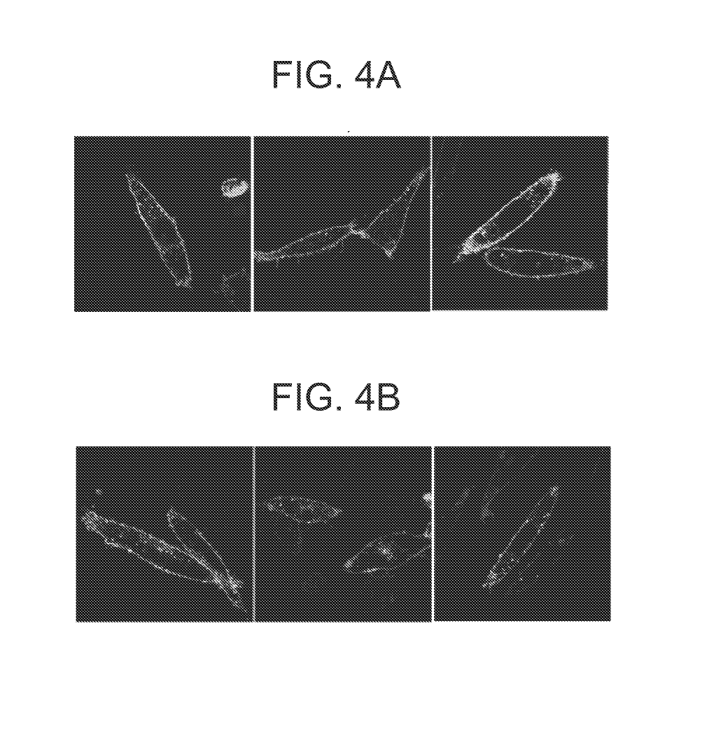

Many GPCRs are internalized by the cell when they are activated. To determine whether GPR139 was internalized when activated, internalization experiments were conducted using an N-terminally V-tagged mouse GPR139. These experiments showed that upon stimulation with L-Trp and L-Phe, VS-tagged GPR139 was internalized (FIG. 4).

About 300 additional phenylalanine analogues have been tested for GPR139 activation using a calcium mobilization (FLIPR®) assay. The results of these experiments indicate that many of these analogues can also activate GPR139. Table 4 lists certain phenylalanine analogues that have been determined to activate GPR139 by calcium mobilization at a concentration of 300 μM (calcium mobilization is expressed as percentage of that observed for L-phenylalanine).

TABLE 4
Phenylalanine derivatives as ligands for GPR139

| | % L-phenylalanine |
|---|---|
| Compound description | at 300 μM (average) |
| 3-Bromo-D-phenylalanine | 172 |
| Styryl-L-alanine | 169 |
| 2-Bromo-L-phenylalanine | 168 |
| 3-Bromo-L-phenylalanine | 167 |
| 3-Nitro-D-Phenylalanine | 164 |
| 2-Naphthyl-L-alanine | 161 |
| 3-Benzothienyl-L-alanine | 161 |
| 1-Naphthyl-D-alanine | 157 |
| (3R)-2-tert-butoxycarbonyl-3,4-dihydro-1H- | 155 |
| isoquinoline-3-carboxylic acid | |
| 2-Naphthyl-D-alanine | 155 |
| 2-(5-bromothienyl)-D-alanine | 154 |
| 4-Bromo-L-phenylalanine | 153 |
| L-1,2,3,4-tetrahydronorharman-3-carboxylic acid | 151 |
| 2-(5-bromothienyl)-L-alanine | 151 |
| 3-Methyl-D-phenylalanine | 150 |
| 4-Trifluoromethyl-L-phenylalanine | 150 |
| 2-Nitro-L-phenylalanine | 150 |
| 4-Methyl-L-phenylalanine | 149 |
| 3,4-Dichloro-L-phenylalanine | 149 |
| 3,4-Dichloro-D-phenylalanine | 149 |
| 3-Chloro-D-phenylalanine | 147 |
| 3-Methyl-L-phenylalanine | 147 |
| 1-Naphthyl-L-alanine | 147 |
| 4-Chloro-L-phenylalanine | 146 |
| 3-Chloro-L-phenylalanine | 145 |
| 2,3,4,5,6-Pentafluoro-D-phenylalanine | 145 |
| 3-Benzothienyl-D-alanine | 143 |
| 4-Bromo-D-phenylalanine | 139 |
| 2,4-Dichloro-D-phenylalanine | 135 |
| 2-Bromo-D-phenylalanine | 135 |
| 2-Methyl-L-phenylalanine | 134 |
| 3-Trifluoromethyl-L-phenylalanine | 133 |
| 4-Fluoro-L-phenylalanine | 132 |
| 3-Nitro-L-phenylalanine | 131 |
| D-2-Amino-5-phenyl-pentanoic acid | 129 |
| 4-Methoxy-L-phenylalanine | 128 |
| 4-Iodo-D-phenylalanine | 125 |
| α-methyl-4-Fluoro-L-phenylalanine | 125 |
| 2,4-Dichloro-L-phenylalanine | 122 |
| 3,4-Difluoro-D-phenylalanine | 121 |
| 2-Fluoro-L-phenylalanine | 119 |
| 3,3-Diphenyl-L-alanine | 117 |
| 4-Iodo-L-phenylalanine | 116 |
| 3-Cyano-L-phenylalanine | 115 |
| 4-tert-Butyl-L-phenylalanine | 115 |
| 3-Fluoro-L-phenylalanine | 114 |
| 2,4-Dimethyl-L-phenylalanine | 113 |
| 4-Nitro-D-phenylalanine | 111 |
| 4-Chloro-D-phenylalanine | 110 |
| 2-Trifluoromethyl-L-phenylalanine | 110 |
| 2-Methyl-D-phenylalanine | 109 |
| d-Homo-phenylalanine | 105 |
| 4-Methyl-D-phenylalanine | 102 |
| 3,5-Difluoro-D-phenylalanine | 102 |
| 2,4-Dimethyl-D-phenylalanine | 101 |
| 2,4-Dinitro-L-phenylalanine | 95 |
| 3-Fluoro-D-phenylalanine | 94 |
| 3-Cyano-D-phenylalanine | 93 |
| 4-Fluoro-D-phenylalanine | 91 |

### Example 2

### Expression Profile of GPR139

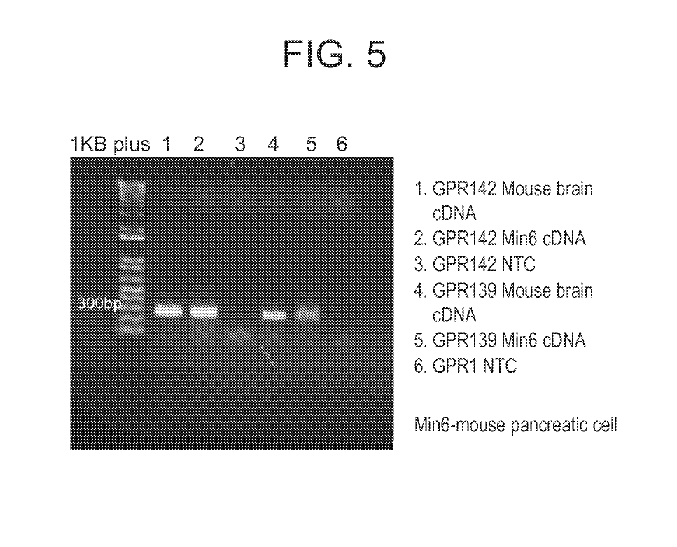

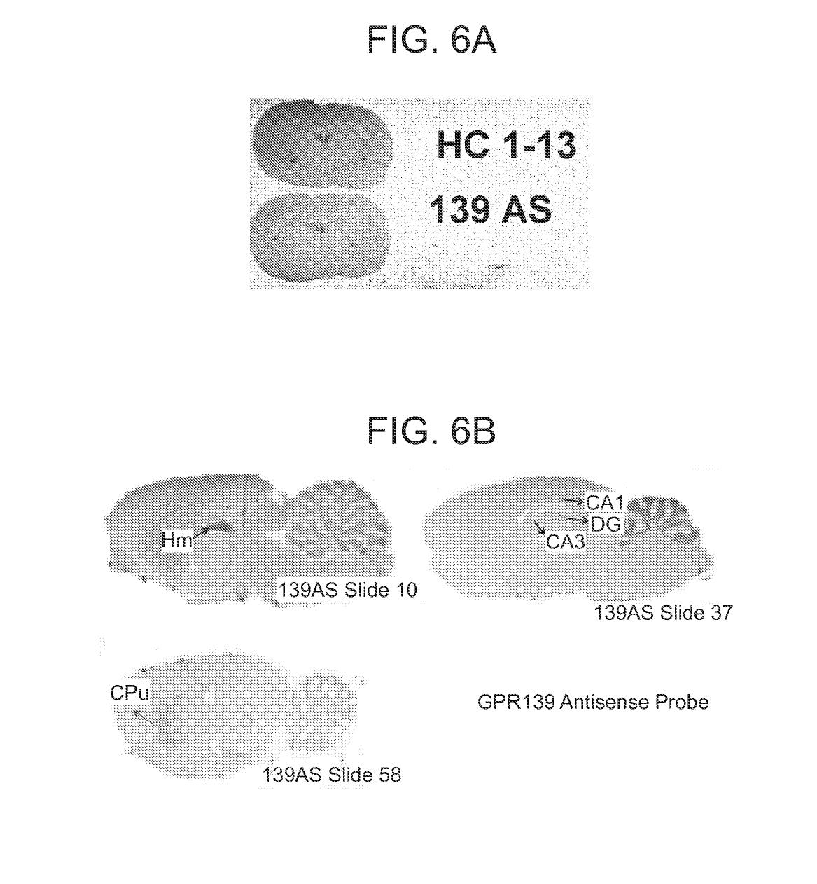

Experiments were conducted to assess tissues or cell types that expressed GPR139. Initial RT-PCR studies focused on assessing expression in mouse brain and mouse pancreatic cells (min6 cells). These studies showed that GPR139 and GPR142 (another orphan GPCR that is closely related to GPR139) are expressed in mouse brain as well as in mouse pancreatic beta cells as detected by RT-PCR (FIG. 5). The expression of GPR139 in rat brain was also confirmed by in situ hybridization (FIGS. 6A and 6B).

What is claimed:

1. A method of activating GPR139 in a subject comprising:
identifying a subject in need of GPR139 activation, and
administering an amount of one or more compounds sufficient to activate GPR139 to the subject,
thereby increasing the level of GPR139 activation relative to its activation level prior to administration of said one or more compounds.

2. The method of claim 1, wherein at least one of said compounds is:
3-Bromo-D-Phenylalanine,
Styryl-L-alanine,
2-Bromo-L-Phenylalanine,
3-Bromo-L-Phenylalanine,
3-Nitro-D-Phenylalanine,
2-Naphthyl-L-alanine,
3-Benzothienyl-L-alanine,
1-Naphthyl-D-alanine,
(3R)-2-tert-butoxycarbony 1-3,4-dihydro-1H-isoquinoline-3-carboxylic acid,
2-Naphthyl-D-alanine,
2-(5-bromothienyl)-D-alanine,
4-Bromo-L-Phenylalanine,
L-1,2,3,4-tetrahydronorharman-3-carboxylic acid,
2-(5-bromothienyl)-L-alanine,
3-Methyl-D-Phenylalanine,
4-Trifluoromethyl-L-Phenylalanine,
2-Nitro-L-Phenylalanine,
4-Methyl-L-Phenylalanine,
3,4-Dichloro-L-Phenylalanine,
3,4-Dichloro-D-Phenylalanine,
3-Chloro-D-Phenylalanine,
3-Methyl-L-Phenylalanine,
1-Naphthyl-L-alanine,
4-Chloro-L-Phenylalanine,
3-Chloro-L-Phenylalanine,
2,3,4,5,6-Pentafluoro-D-Phenylalanine,
3-Benzothienyl-D-alanine,
4-Bromo-D-Phenylalanine,
2,4-Dichloro-D-Phenylalanine,
2-Bromo-D-Phenylalanine,
2-Methyl-L-Phenylalanine,
3-Trifluoromethyl-L-Phenylalanine,
4-Fluoro-L-Phenylalanine,
3-Nitro-L-Phenylalanine,
D-2-Amino-5-phenyl-pentanoic acid,
4-Methoxy-L-Phenylalanine,
4-Iodo-D-Phenylalanine,
α-methyl-4-Fluoro-L-Phenylalanine,
2,4-Dichloro-L-Phenylalanine,
3,4-Difluoro-D-Phenylalanine,
2-Fluoro-L-Phenylalanine,
3,3-Diphenyl-L-alanine,
4-Iodo-L-Phenylalanine,
3-Cyano-L-Phenylalanine,
4-tert-Butyl-L-Phenylalanine,
3-Fluoro-L-Phenylalanine,
2,4-Dimethyl-L-Phenylalanine,
4-Nitro-D-Phenylalanine,
4-Chloro-D-Phenylalanine,
2-Trifluoromethyl-L-Phenylalanine,
2-Methyl-D-Phenylalanine,
d-Homo-Phenylalanine,
4-Methyl-D-Phenylalanine,
3,5-Difluoro-D-Phenylalanine,
2,4-Dimethyl-D-Phenylalanine,
2,4-Dinitro-L-Phenylalanine,
3-Fluoro-D-Phenylalanine,
3-Cyano-D-Phenylalanine, or
4-Fluoro-D-Phenylalanine.

3. The method of claim 1, wherein at least one of said compounds is L-tryptophan, L-phenylalanine, tryptamine, amphetamine, D-tryptophan, D-phenylalanine or β-phenylethylamine.

4. The method of any one of claims 1 to 3, wherein the compound is administered locally to the subject.

5. The method of any one of claims 1 to 3, wherein the compound is administered orally or intravenously to the subject.

6. A method for detecting GPR139 activation comprising, exposing a cell or a membrane obtained from the cell that expressed GPR139 to a compound, and determining whether or not GPR139 is activated by the compound.

7. A method as defined in claim 6, wherein the cell line is modified to recombinantly express GPR139.

8. A method as defined in claim 6 further comprising, wherein the cell is obtained by co-expressing GO2Q chimeric G-protein and GPR139 in a cell.

9. A method as defined in claim 6, wherein the compound is selected from the group consisting of:
3-Bromo-D-Phenylalanine;
Styryl-L-alanine;
2-Bromo-L-Phenylalanine;
3-Bromo-L-Phenylalanine;
3-Nitro-D-Phenylalanine;
2-Naphthyl-L-alanine;
3-Benzothienyl-L-alanine;
1-Naphthyl-D-alanine;
(3R)-2-tert-butoxycarbony 1-3,4-dihydro-1H-isoquinoline-3-carboxylic acid;
2-Naphthyl-D-alanine;
2-(5-bromothienyl)-D-alanine;
4-Bromo-L-Phenylalanine;
L-1,2,3,4-tetrahydronorharman-3-carboxylic acid;
2-(5-bromothienyl)-L-alanine;
3-Methyl-D-Phenylalanine;
4-Trifluoromethyl-L-Phenylalanine;
2-Nitro-L-Phenylalanine;
4-Methyl-L-Phenylalanine;
3,4-Dichloro-L-Phenylalanine;
3,4-Dichloro-D-Phenylalanine;
3-Chloro-D-Phenylalanine;
3-Methyl-L-Phenylalanine;
1-Naphthyl-L-alanine;
4-Chloro-L-Phenylalanine;
3-Chloro-L-Phenylalanine;
2,3,4,5,6-Pentafluoro-D-Phenylalanine;
3-Benzothienyl-D-alanine;
4-Bromo-D-Phenylalanine;
2,4-Dichloro-D-Phenylalanine;
2-Bromo-D-Phenylalanine;
2-Methyl-L-Phenylalanine;
3-Trifluoromethyl-L-Phenylalanine;
4-Fluoro-L-Phenylalanine;
3-Nitro-L-Phenylalanine;
D-2-Amino-5-phenyl-pentanoic acid;
4-Methoxy-L-Phenylalanine;
4-Iodo-D-Phenylalanine;
α-methyl-4-Fluoro-L-Phenylalanine;
2,4-Dichloro-L-Phenylalanine,
3,4-Difluoro-D-Phenylalanine;
2-Fluoro-L-Phenylalanine;
3,3-Diphenyl-L-alanine;
4-Iodo-L-Phenylalanine;
3-Cyano-L-Phenylalanine;
4-tert-Butyl-L-Phenylalanine;
3-Fluoro-L-Phenylalanine;
2,4-Dimethyl-L-Phenylalanine;
4-Nitro-D-Phenylalanine;
4-Chloro-D-Phenylalanine;
2-Trifluoromethyl-L-Phenylalanine;
2-Methyl-D-Phenylalanine;
d-Homo-Phenylalanine;
4-Methyl-D-Phenylalanine;
3,5-Difluoro-D-Phenylalanine;
2,4-Dimethyl-D-Phenylalanine;
2,4-Dinitro-L-Phenylalanine;
3-Fluoro-D-Phenylalanine;
3-Cyano-D-Phenylalanine;
4-Fluoro-D-Phenylalanine;
L-tryptophan;
L-phenylalanine;
Tryptamine;
Amphetamine;
D-tryptophan;
D-phenylalanine; and
β-phenylethylamine.

10. A method of modulating a neurological function, comprising contacting a brain cell with at least one compound that is:
3-Bromo-D-Phenylalanine,
Styryl-L-alanine,
2-Bromo-L-Phenylalanine,
3-Bromo-L-Phenylalanine,
3-Nitro-D-Phenylalanine,
2-Naphthyl-L-alanine,
3-Benzothienyl-L-alanine,
1-Naphthyl-D-alanine,
(3R)-2-tert-butoxycarbony 1-3,4-dihydro-1H-isoquinoline-3-carboxylic acid,
2-Naphthyl-D-alanine,
2-(5-bromothienyl)-D-alanine,
4-Bromo-L-Phenylalanine,
L-1,2,3,4-tetrahydronorharman-3-carboxylic acid,
2-(5-bromothienyl)-L-alanine,
3-Methyl-D-Phenylalanine,
4-Trifluoromethyl-L-Phenylalanine,
2-Nitro-L-Phenylalanine,
4-Methyl-L-Phenylalanine,
3,4-Dichloro-L-Phenylalanine,
3,4-Dichloro-D-Phenylalanine,
3-Chloro-D-Phenylalanine,
3-Methyl-L-Phenylalanine,
1-Naphthyl-L-alanine,
4-Chloro-L-Phenylalanine,
3-Chloro-L-Phenylalanine,
2,3,4,5,6-Pentafluoro-D-Phenylalanine,
3-Benzothienyl-D-alanine,
4-Bromo-D-Phenylalanine,
2,4-Dichloro-D-Phenylalanine,
2-Bromo-D-Phenylalanine,
2-Methyl-L-Phenylalanine,
3-Trifluoromethyl-L-Phenylalanine,
4-Fluoro-L-Phenylalanine,
3-Nitro-L-Phenylalanine,
D-2-Amino-5-phenyl-pentanoic acid,
4-Methoxy-L-Phenylalanine,
4-Iodo-D-Phenylalanine,
α-methyl-4-Fluoro-L-Phenylalanine,
2,4-Dichloro-L-Phenylalanine,
3,4-Difluoro-D-Phenylalanine,
2-Fluoro-L-Phenylalanine,
3,3-Diphenyl-L-alanine,
4-Iodo-L-Phenylalanine,
3-Cyano-L-Phenylalanine,
4-tert-Butyl-L-Phenylalanine,
3-Fluoro-L-Phenylalanine,
2,4-Dimethyl-L-Phenylalanine,
4-Nitro-D-Phenylalanine,
4-Chloro-D-Phenylalanine,
2-Trifluoromethyl-L-Phenylalanine,
2-Methyl-D-Phenylalanine,
d-Homo-Phenylalanine,
4-Methyl-D-Phenylalanine,
3,5-Difluoro-D-Phenylalanine,
2,4-Dimethyl-D-Phenylalanine,
2,4-Dinitro-L-Phenylalanine,
3-Fluoro-D-Phenylalanine,
3-Cyano-D-Phenylalanine, or
4-Fluoro-D-Phenylalanine,
and thereby increasing the level of GPR139 activation in the cell.

11. A method of modulating a neurological function, comprising contacting a brain cell with at least one compound that is L-tryptophan, L-phenylalanine, tryptamine, amphetamine, D-tryptophan, D-phenylalanine or β-phenylethylamine.

12. The method of claim 10 or 11, wherein the compound is administered locally to a subject having a neuronal cell.

13. The method of claim 10 or 11, wherein the compound is administered orally or intravenously to a subject having a neuronal cell.

14. A method of modulating Phenylketonuria (PKU), and pituitary dysfunctions, comprising contacting a cell with at least one compound that is:
3-Bromo-D-Phenylalanine,
Styryl-L-alanine,
2-Bromo-L-Phenylalanine,
3-Bromo-L-Phenylalanine,
3-Nitro-D-Phenylalanine,
2-Naphthyl-L-alanine,
3-Benzothienyl-L-alanine,
1-Naphthyl-D-alanine,
(3R)-2-tert-butoxycarbony 1-3,4-dihydro-1H-isoquinoline-3-carboxylic acid,
2-Naphthyl-D-alanine,
2-(5-bromothienyl)-D-alanine,
4-Bromo-L-Phenylalanine,
L-1,2,3,4-tetrahydronorharman-3-carboxylic acid,
2-(5-bromothienyl)-L-alanine,
3-Methyl-D-Phenylalanine,
4-Trifluoromethyl-L-Phenylalanine,
2-Nitro-L-Phenylalanine,
4-Methyl-L-Phenylalanine,
3,4-Dichloro-L-Phenylalanine,
3,4-Dichloro-D-Phenylalanine,
3-Chloro-D-Phenylalanine,
3-Methyl-L-Phenylalanine,
1-Naphthyl-L-alanine,
4-Chloro-L-Phenylalanine,
3-Chloro-L-Phenylalanine,
2,3,4,5,6-Pentafluoro-D-Phenylalanine,
3-Benzothienyl-D-alanine,
4-Bromo-D-Phenylalanine,
2,4-Dichloro-D-Phenylalanine,
2-Bromo-D-Phenylalanine,
2-Methyl-L-Phenylalanine,
3-Trifluoromethyl-L-Phenylalanine,
4-Fluoro-L-Phenylalanine,
3-Nitro-L-Phenylalanine,
D-2-Amino-5-phenyl-pentanoic acid,
4-Methoxy-L-Phenylalanine,
4-Iodo-D-Phenylalanine,
α-methyl-4-Fluoro-L-Phenylalanine,
2,4-Dichloro-L-Phenylalanine,
3,4-Difluoro-D-Phenylalanine,
2-Fluoro-L-Phenylalanine,
3,3-Diphenyl-L-alanine,
4-Iodo-L-Phenylalanine,
3-Cyano-L-Phenylalanine,
4-tert-Butyl-L-Phenylalanine,
3-Fluoro-L-Phenylalanine,
2,4-Dimethyl-L-Phenylalanine,
4-Nitro-D-Phenylalanine,
4-Chloro-D-Phenylalanine,
2-Trifluoromethyl-L-Phenylalanine,
2-Methyl-D-Phenylalanine,
d-Homo-Phenylalanine,
4-Methyl-D-Phenylalanine,
3,5-Difluoro-D-Phenylalanine,
2,4-Dimethyl-D-Phenylalanine,
2,4-Dinitro-L-Phenylalanine,
3-Fluoro-D-Phenylalanine,
3-Cyano-D-Phenylalanine, or
4-Fluoro-D-Phenylalanine,
and thereby increasing the level of GPR139 activation in the cell.

15. A method of modulating a Phenylketonuria (PKU), and pituitary dysfunctions, comprising contacting a cell with at least one compound that is L-tryptophan, L-phenylalanine, tryptamine, amphetamine, D-tryptophan, D-phenylalanine or β-phenylethylamine.
